(19) United States
(12) Patent Application Publication
(10) Pub. No.: US 2024/0031468 A1
(43) Pub. Date: Jan. 25, 2024

(54) RECOMMENDED ACTION SELECTION APPARATUS, RECOMMENDED ACTION SELECTION METHOD, AND RECOMMENDED ACTION SELECTION PROGRAM

(71) Applicant: NIPPON TELEGRAPH AND TELEPHONE CORPORATION, Tokyo (JP)

(72) Inventors: Tae SATO, Musashino-shi, Tokyo (JP); Hitoshi SESHIMO, Musashino-shi, Tokyo (JP); Reiko ARUGA, Musashino-shi, Tokyo (JP); Akihiro CHIBA, Musashino-shi, Tokyo (JP)

(73) Assignee: NIPPON TELEGRAPH AND TELEPHONE CORPORATION, Tokyo (JP)

(21) Appl. No.: 18/024,826

(22) PCT Filed: Sep. 11, 2020
(86) PCT No.: PCT/JP2020/034399

§ 371 Date: Mar. 6, 2023

(51) Int. Cl. H04M 1/72403

(52) U.S. Cl. CPC H04M 1/72403

(57) ABSTRACT

A recommended action selection device according to an embodiment includes: a user non-recommended action detection unit that detects that a user is taking a non-recommended action; an execution positive factor collection unit that collects an execution positive factor that is a subjective factor for which the user is taking the non-recommended action; and a recommended action selection unit that selects a subjective factor other than the execution positive factor, acquires a plurality of recommended action possibilities, and selects a recommended action on the basis of a first score indicating how easily each of the plurality of recommended action possibilities is implemented by the user for the subjective factor selected, a second score indicating how familiar the user is with each of the plurality of recommended action possibilities, and a first objective value for an evaluation axis for evaluating the plurality of recommended action possibilities.

### TECHNICAL FIELD

The present invention relates to a recommended action selection device, a recommended action selection method, and a recommended action selection program.

### BACKGROUND ART

It is important to incorporate actions recommended by doctors and public health nurses into our daily lives in the prevention of the onset and aggravation of lifestyle-related diseases. However, simply knowing a recommended action may not lead to motivation for the action.

Thus, for example, Non Cited Literature 1 discloses a technology of presenting an exercise menu with a variety of exercise intensity that users do not get bored and are able to do the exercise menu using exercise information.

### CITATION LIST

### Non Patent Literature

Non Patent Literature 1: Ayu Hoshino, Hiroshi Takenouchi, Masataka Tokumaru “Healthcare system that suggests personalized exercises and meals”, Proceedings of the Fuzzy System Symposium, Japan Society for Fuzzy Theory and Intelligent Informatics, 2015, Vol. 31, TE1-2

### SUMMARY OF INVENTION

### Technical Problem

However, Non Patent Literature 1 does not consider a subjective merit of a recommended action, that is, enhancement of a merit for a user, which is different for each of users.

An object of the present invention is to enable selection of a recommended action that is likely to cause a user to feel that there is a merit.

### Solution to Problem

To solve the above problem, a recommended action selection device of the present invention includes: a user non-recommended action detection unit that detects that a user is taking a non-recommended action; an execution positive factor collection unit that collects an execution positive factor that is a subjective factor for which the user is taking the non-recommended action; and a recommended action selection unit that selects a subjective factor other than the execution positive factor, acquires a plurality of recommended action possibilities, and selects a recommended action to be recommended to the user on the basis of a first score indicating how easily each of the plurality of recommended action possibilities is implemented by the user for the subjective factor selected, a second score indicating how familiar the user is with each of the plurality of recommended action possibilities, and a first objective value for an evaluation axis for evaluating the plurality of recommended action possibilities.

### Advantageous Effects of Invention

According to an aspect of the present invention, it is possible to select a recommended action that is likely to cause a user to feel that there is a merit.

### BRIEF DESCRIPTION OF DRAWINGS

FIG. 1 is a schematic diagram illustrating an example of a user according to an embodiment of the present invention and a user terminal that is a recommended action selection device.

FIG. 2 is a block diagram illustrating an example of a hardware configuration of the user terminal.

FIG. 3 is a block diagram illustrating a functional configuration of the user terminal in the embodiment.

FIG. 4 is a flowchart illustrating an example of recommended action selection operation of the user terminal in the present embodiment.

FIG. 5 is a flowchart illustrating an example of more detailed operation of step S103.

FIG. 6 is a diagram illustrating an example of an objective value of a recommended action, a score of familiarity, and a score of a subjective factor, for each of recommended actions.

FIG. 7A is a diagram illustrating an example of message syntax stored in a message syntax database.

FIG. 7B is a diagram illustrating an example of a message generated by a message generation unit.

### DESCRIPTION OF EMBODIMENTS

Hereinafter, embodiments according to the present invention will be described with reference to the drawings.

[Configuration]

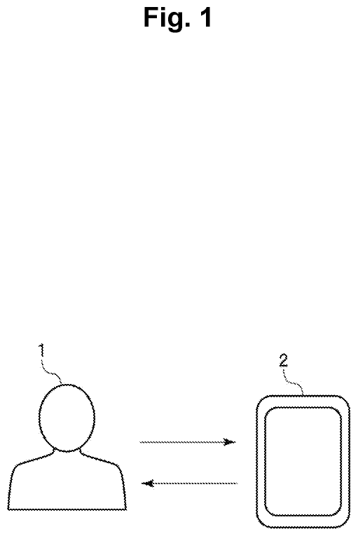

FIG. 1 is a schematic diagram illustrating an example of a user 1 according to an embodiment of the present invention and a user terminal 2 that is a recommended action selection device.

The user terminal 2 is a portable terminal such as a smartphone, a tablet terminal, or a wearable terminal. Although only one user terminal 2 is illustrated in FIG. 1 for simplification of the drawing, a large number of user terminals may be included. For example, a first user terminal such as a smartphone receives and processes information from a base station or the like, and then transmits the processed information to a second user terminal such as a wearable terminal. Then, the second user terminal can display a message to the user on the basis of the received information.

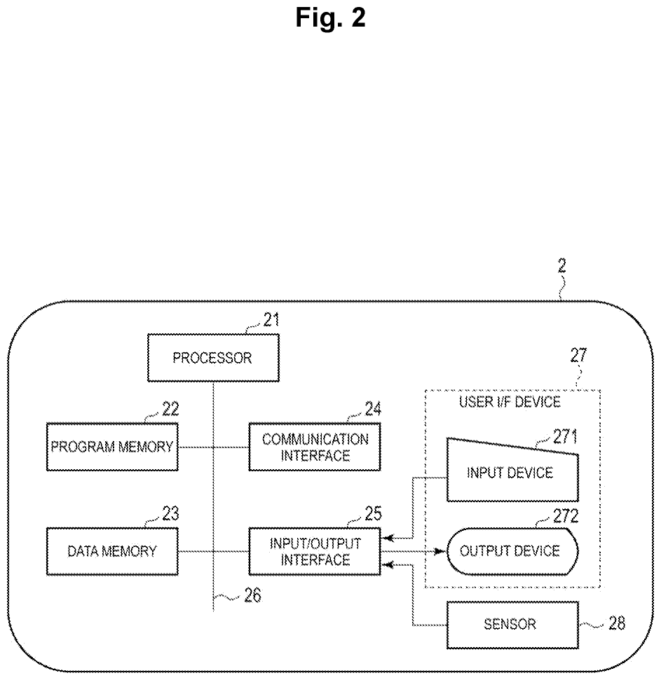

FIG. 2 is a block diagram illustrating an example of a hardware configuration of the user terminal 2.

The user terminal 2 includes, for example, a hardware processor 21 such as a central processing unit (CPU) or a micro processing unit (MPU). In addition, a program memory 22, a data memory 23, a communication interface 24, and an input/output interface 25 are connected to the processor 21 via a bus 26.

The program memory 22 can use, as a storage medium, a combination of a nonvolatile memory to and from which writing and reading can be performed at any time, such as an erasable programmable read only memory (EPROM) or a memory card, and a nonvolatile memory such as a read only memory (ROM), for example. The program memory 22 stores programs necessary for executing various types of processing, which include a notification control program. That is, any processing function unit in each unit of a functional configuration described later can be implemented by the above-described processor 21 reading and executing a program stored in the program memory 22.

The data memory 23 is a storage using, as a storage medium, a combination of a nonvolatile memory to and from which writing and reading can be performed at any time, such as a memory card, and a volatile memory such as a random access memory (RAM), for example. The data memory 23 is used to store data acquired and generated in the process in which the processor 21 executes a program to perform various types of processing.

The communication interface 24 includes one or a plurality of wireless communication modules. For example, the communication interface 24 includes a wireless communication module wirelessly connected to a Wi-Fi access point or a mobile phone base station. Furthermore, the communication interface 24 includes a wireless communication module for wirelessly connecting to another user terminal using a short-distance wireless technology. Under the control of the processor 21, the wireless communication module can communicate with a mobile phone base station or the like to transmit and receive various types of information. Note that the communication interface 24 may include one or a plurality of wired communication modules.

The input/output interface 25 is an interface with a user interface device 27. Note that, in FIG. 2, the “user interface device” is described as “user IF device”.

The user interface device 27 includes an input device 271 and an output device 272. The input device 271 is, for example, an input detection sheet that is disposed on a display screen of a display device as the output device 272 and employs an electrostatic method or a pressure method, and outputs a touch position of the user to the processor 21 via the input/output interface 25. The output device 272 is a display device using, for example, liquid crystal, organic electro luminescence (EL), or the like, and displays an image and a message according to a signal input from the input/output interface 25.

The sensor 28 includes, for example, an acceleration sensor, a proximity sensor, and the like for detecting an action of the user. Furthermore, the sensor 28 includes a global positioning system (GPS) receiver for detecting a position of the user terminal 2. Note that the processor 21 can also acquire position information of the user terminal 2 by use of the signal strength of a Wi-Fi access point or a mobile phone wireless base station used by the communication interface 24, a Bluetooth (registered trademark) beacon, or the like. Therefore, the sensor 28 does not have to include the GPS receiver. In addition, without including the sensor 28 itself, the user terminal 2 may capture sensor data acquired by an external sensor via the communication interface 24.

(1) Functional Configuration

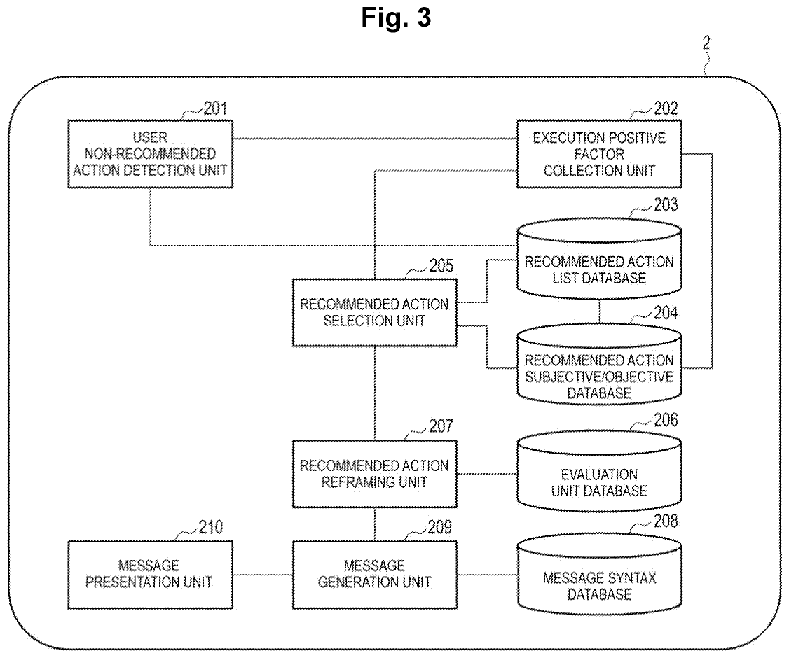

FIG. 3 is a block diagram illustrating a functional configuration of the user terminal 2 in the embodiment.

The user terminal 2 includes a user non-recommended action detection unit 201, an execution positive factor collection unit 202, a recommended action list database 203, a recommended action subjective/objective database 204, a recommended action selection unit 205, an evaluation unit database 206, a recommended action reframing unit 207, a message syntax database 208, a message generation unit 209, and a message presentation unit 210. Here, the user non-recommended action detection unit 201, the execution positive factor collection unit 202, the recommended action selection unit 205, the recommended action reframing unit 207, the message generation unit 209, and the message presentation unit 210 are processing function units implemented by the processor 21 reading and executing a recommended action selection program stored in the program memory 22. In addition, the recommended action list database 203, the recommended action subjective/objective database 204, the evaluation unit database 206, and the message syntax database 208 can be provided in the data memory 23, for example.

The user non-recommended action detection unit 201 detects that the user 1 is performing or is about to perform a non-recommended action that is not recommended for the user 1. In a case where the user 1 aims to increase calorie consumption, the non-recommended action refers to an action that consumes less calories, for example, sitting on a chair or lying down. For example, when the user 1 sets a target, the user terminal 2 acquires recommended actions and non-recommended actions by communicating with a server or the like not illustrated in FIG. 1 using the communication interface 24, and stores the recommended actions and the non-recommended actions in the recommended action list database 203 in advance. For example, the user non-recommended action detection unit 201 estimates a current action of the user 1 on the basis of the sensor data of the sensor 28 of the user terminal 2, and if the user 1 is performing a non-recommended action stored in the recommended action list database 203, the action is detected.

When detecting by the user non-recommended action detection unit 201 that the user 1 is performing a non-recommended action, the execution positive factor collection unit 202 acquires a plurality of subjective factors considered to be inducing a non-recommended action, from the recommended action subjective/objective database 204. A subjective factor in a case where the user non-recommended action detection unit 201 detects the non-recommended action is a subjective factor of the user 1, for example, that the user 1 likes to perform the non-recommended action, that it is comfortable or easy for the user 1 to perform the non-recommended action, or the like. Then, for example, the execution positive factor collection unit 202 presents the acquired plurality of subjective factors to the user 1 via the output device 272 of the user interface device 27, and collects an execution positive factor that is a subjective factor causing the user 1 to take a non-recommended action, via the input device 271. Note that the execution positive factor collection unit 202 may collect the execution positive factor by displaying the acquired plurality of subjective factors in a selection format and causing the user 1 to make a selection. Alternatively, the execution positive factor collection unit 202 may have the user 1 directly input a subjective factor, and may collect a subjective factor corresponding to a result of the input as the execution positive factor.

The recommended action list database 203 is a database that stores recommended actions and non-recommended actions as a list. The recommended action is an action that the user 1 is recommended to practice, and is, for example, stepping, stretching, walking, jogging, swimming, or the like in a case where an increase in calorie consumption is targeted. In that case, as described above, the non-recommended action refers to, for example, sitting on a chair, lying down, or the like. In addition, it is a matter of course that the recommended action and the non-recommended action can be added or reduced by input from the user 1 via the user interface device 27.

The recommended action subjective/objective database 204 stores the subjective factors. Furthermore, the recommended action subjective/objective database 204 stores an objective value for an evaluation axis for evaluating each recommended action. In a case where the evaluation axis for evaluating the recommended action is calorie consumption, the objective value is, for example, calorie consumption per unit time. In addition, the recommended action subjective/objective database 204 stores a score indicating how familiar the user 1 is with each recommended action stored in the recommended action list database 203 and a score indicating how easily each recommended action for a subjective factor is implemented by the user 1. The score indicating how familiar the user 1 is represents familiarity indicating how familiar each recommended action is to the user 1. The score indicating how easily each recommended action for a subjective factor is implemented by the user 1 may be a score set in advance by the user 1, or a question about how easily each recommended action for a subjective factor is implemented may be presented to the user 1 via the output device 272 at a timing when the sensor 28 of the user terminal 2 detects that the recommended action is executed by using the sensor data of the sensor 28, and an answer may be collected from the user 1 via the input device 271. Note that, although the recommended action list database 203 and the recommended action subjective/objective database 204 are described as separate databases, it is a matter of course that they can be a single database.

The recommended action selection unit 205 calculates an objective value for an evaluation axis for evaluating a non-recommended action. For example, the recommended action selection unit 205 calculates an objective value for an evaluation axis for evaluating a non-recommended action with reference to data stored in the recommended action subjective/objective database 204. The recommended action selection unit 205 acquires a plurality of recommended action possibilities from the recommended action list database 203. The recommended action selection unit 205 randomly selects one subjective factor other than the execution positive factor collected by the execution positive factor collection unit 202 from the recommended action subjective/objective database 204. Furthermore, the recommended action selection unit 205 determines a recommended action from the plurality of recommended action possibilities on the basis of a first score indicating how easily each of the plurality of recommended action possibilities is implemented by the user 1 for the subjective factor selected, a second score indicating how familiar the user 1 is with each of the plurality of recommended action possibilities, and an objective value for an evaluation axis for evaluating the plurality of recommended action possibilities. Note that a more detailed method of determining the recommended action will be described later.

The evaluation unit database 206 is a database that stores evaluation units when utility regarding a non-recommended action and a recommended action is presented to the user 1 numerically.

The recommended action reframing unit 207 converts the objective value for the evaluation axis for evaluating the non-recommended action and the recommended action into an objective value of a presentation evaluation unit stored in the evaluation unit database 206. Furthermore, the recommended action reframing unit 207 calculates the utility of the recommended action on the basis of the converted objective value of the non-recommended action and the converted objective value of the recommended action.

The message syntax database 208 stores message syntax for generating a message by the message generation unit 209.

The message generation unit 209 refers to the message syntax stored in the message syntax database 208, and generates a message on the basis of the non-recommended action, the evaluation unit, the converted objective value of the recommended action, the evaluation axis, the selected subjective factor, the selected recommended action, and the calculated utility.

The message presentation unit 210 presents the message generated by the message generation unit 209 to the user 1 via the user interface device 27.

(2) Operation

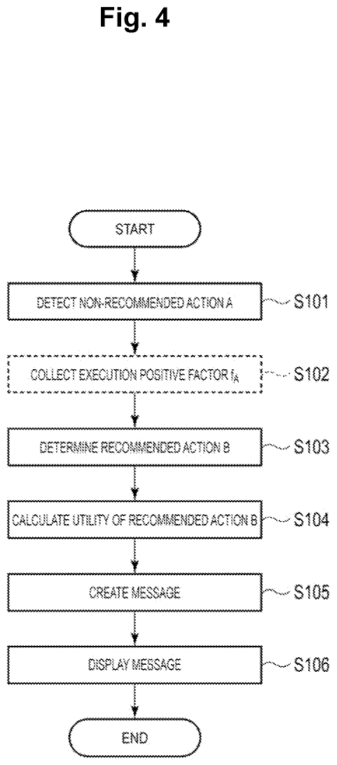

FIG. 4 is a flowchart illustrating an example of recommended action selection operation of the user terminal 2 in the present embodiment. The processor 21 of the user terminal 2 reads and executes the recommended action selection program stored in the program memory 22, whereby the operation of the flowchart is implemented.

For example, it is assumed that the user 1 aims to increase the calorie consumption. In this case, the flowchart starts at regular time intervals. Alternatively, the flowchart may be started by a user instruction from the input device 271 when the user 1 tries to take some action. Note that it is assumed that the sensor data acquired by the sensor 28 is accumulated in the data memory 23 every time the sensor data is acquired.

The user non-recommended action detection unit 201 of the user terminal 2 detects that the user 1 is taking an action (non-recommended action A) that is not recommended for the user 1 on the basis of sensor data of the acceleration sensor and the like (step S101). For example, the user non-recommended action detection unit 201 detects that the user 1 lies down at home for many hours. The user non-recommended action detection unit 201 notifies the execution positive factor collection unit 202 that the user 1 is taking the non-recommended action A.

The execution positive factor collection unit 202 collects an execution positive factor f_{A} on the basis of notification from the user non-recommended action detection unit 201 (step S102). Specifically, upon receiving the notification from the user non-recommended action detection unit 201, the execution positive factor collection unit 202 acquires a plurality of subjective factors considered to be inducing the non-recommended action A, from the recommended action subjective/objective database 204. Then, the execution positive factor collection unit 202 presents the acquired plurality of subjective factors to the user 1 via the output device 272 of the user interface device 27, and acquires the execution positive factor f_{A} that is a subjective factor causing the user 1 input via the input device 271 to take the non-recommended action A. The execution positive factor collection unit 202 transmits, to the recommended action selection unit 205, execution positive factor information including information on the execution positive factor f_{A} acquired together with the non-recommended action A in the notification. Note that the execution positive factor collection unit 202 can also collect the execution positive factor f_{A} from the user 1 in advance. In this case, upon receiving the notification from the user non-recommended action detection unit 201 in step S102, the execution positive factor collection unit 202 transmits, to the recommended action selection unit 205, execution positive factor information including information on the execution positive factor f_{A} acquired in advance and the non-recommended action A.

Upon receiving the execution positive factor information from the user non-recommended action detection unit 201, the recommended action selection unit 205 selects a recommended action B (step S103). Here, one or a plurality of recommended actions B may be selected.

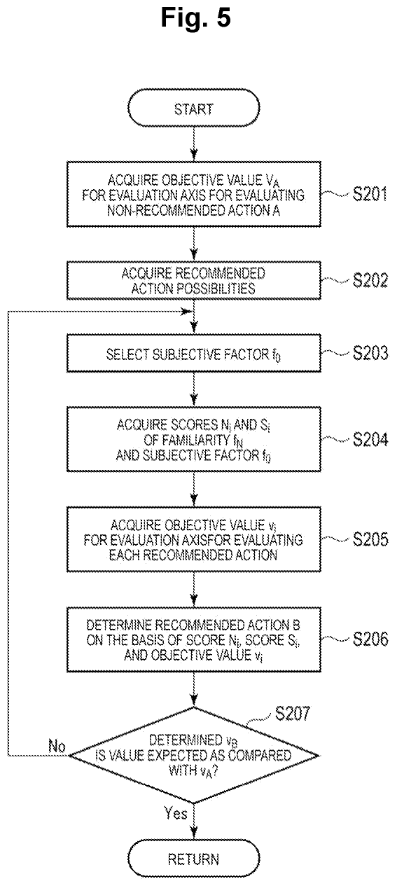

FIG. 5 is a flowchart illustrating an example of more detailed operation of step S103.

The recommended action selection unit 205 refers to the data stored in the recommended action subjective/objective database 204, and calculates an objective value v_{A} for an evaluation axis for evaluating the non-recommended action A included in the received execution positive factor information (step S201). As described above, since the user 1 aims to increase the calorie consumption, the evaluation axis is the calorie consumption. For that reason, the objective value v_{A} is, for example, calorie consumption per unit time in a case where the non-recommended action A is performed. Here, it is a matter of course that the unit time may be an arbitrary time.

The recommended action selection unit 205 acquires n recommended action possibilities from the recommended action list database 203 (step S202). Here, n is an integer of greater than or equal to 1.

The recommended action selection unit 205 randomly selects a subjective factor f₀ other than the execution positive factor f_{A} included in the received execution positive factor information from the subjective factors stored in the recommended action subjective/objective database 204 (step S203). The selected subjective factor f₀ is for regrasping the recommended action from a viewpoint different from the execution positive factor f_{A}, and is for causing the user 1 to turn one's attention to another way of grasping and to recognize merit.

The recommended action selection unit 205 acquires, from the recommended action subjective/objective database 204, a score Nᵢ of familiarity f N and a score Sᵢ of the subjective factor f₀ for each of the plurality of recommended action possibilities acquired from the recommended action list database 203 (step S204). Here, i is any variable from 1 to n (the number of recommended action possibilities).

The recommended action selection unit 205 acquires an objective value vᵢ for an evaluation axis for evaluating each of the plurality of recommended action possibilities from the recommended action subjective/objective database 204 (step S205). Here, the same evaluation axis as the evaluation axis used in step S201 is used for the objective value vᵢ. Thus, the objective value vᵢ represents calorie consumption per unit time in a case where the recommended action is performed.

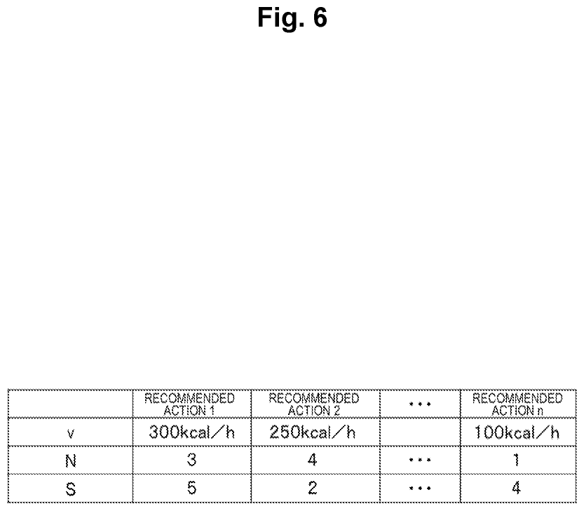

FIG. 6 is a diagram illustrating an example of the objective value vᵢ of a recommended action, the score Nᵢ of the familiarity f_{N}, and the score Sᵢ of the subjective factor f₀, for each of recommended actions. Note that the objective value vᵢ indicated in FIG. 6 represents calorie consumption per hour. In addition, it is assumed that all these values are stored in the recommended action subjective/objective database 204.

The recommended action selection unit 205 determines the recommended action B on the basis of the following equations using the score Nᵢ of the familiarity f_{N}, the score Sᵢ of the subjective factor f₀, and the objective value vᵢ of the recommended action acquired (step S206).

B=max({b₁,b₂, . . . ,bₙ})

bᵢ=w_{N}Nᵢ+wₛSᵢ+wᵥvᵢ (i=1,2, . . . ,n)

Here, the function max( ) is a function that returns an index of an element having the maximum value among the elements bᵢ, and w_{N}, wₛ, and wᵥ are predetermined weights. The weights may be weights for respectively normalizing N Sᵢ, and vᵢ, or may be weights for adjustment depending on elements that are strongly desired to work. In a case where a plurality of the recommended actions B is determined, the function max( ) is a function that returns indexes of a desired number of elements in order from the maximum value of the values of the respective elements bᵢ. The equations make it easy for the user 1 to select a familiar recommended action among the plurality of recommended action possibilities. As a result, the user 1 can easily grasp the recommended action as an action in the life of the user 1. In addition, from the above equations, the recommended action selection unit 205 selects, as the recommended action B, a recommended action possibility having the maximum sum of the score Nᵢ of the familiarity f_{N}, the score Sᵢ of the subjective factor f₀, and the objective value vᵢ that are normalized or weighted.

The recommended action selection unit 205 determines whether or not an objective value vs for the evaluation axis for evaluating the selected recommended action B has a value expected as compared with the objective value v_{A} for the evaluation axis for evaluating the non-recommended action A (step S207). For example, for the purpose of increasing the calorie consumption, if the objective value v s of the recommended action B is larger than the objective value v_{A} of the execution positive factor f_{A}, the calorie consumption increases, so that the objective value vs of the recommended action B has the value expected. In a case where the objective value vs of the recommended action B has the value expected, the recommended action selection unit 205 transmits recommended action selection information including information on the non-recommended action A, the objective value v_{A}, the recommended action B, the objective value vs, the evaluation axis, and the subjective factor f₀ to the recommended action reframing unit 207. Thereafter, step S103 ends, and processing returns to the upper routine. In a case where the objective value vs of the selected recommended action B does not have the value expected, the processing returns to step S203. Thereafter, the recommended action selection unit 205 selects another subjective factor and determines a recommended action.

The recommended action reframing unit 207 calculates utility of the recommended action B on the basis of the objective value v_{A} and the objective value vs included in the received recommended action selection information (step S104). Specifically, the recommended action reframing unit 207 refers to the presentation evaluation unit registered in advance in the evaluation unit database 206, and converts the objective value v_{A} and the objective value vs into objective values for the presentation evaluation unit. The presentation evaluation unit is an arbitrary time unit such as 5 minutes or 10 minutes. Furthermore, the recommended action reframing unit 207 calculates the utility of the recommended action B by dividing the objective value vs converted into the presentation evaluation unit by the objective value v_{A}. For example, in a case where the non-recommended action A is that the user 1 is lying down, and the converted objective value v_{A} is calorie consumption of 10 kcal every 10 minutes, and the recommended action B is stepping, and the converted objective value vs is calorie consumption of 50 kcal every minutes, the utility of the recommended action B is increased by 5 times. The recommended action reframing unit 207 transmits, to the message generation unit 209, message creation information including information on the objective value v_{A} converted into the presentation evaluation unit, the non-recommended action A, the recommended action B converted into the presentation evaluation unit, the subjective factor f₀, the evaluation axis, the presentation evaluation unit, and the calculated utility.

The message generation unit 209 refers to the message syntax stored in the message syntax database 208, and generates a message on the basis of the received message creation information (step S105).

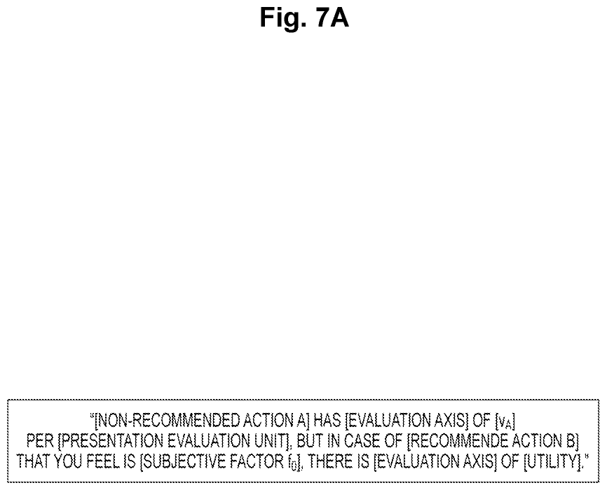

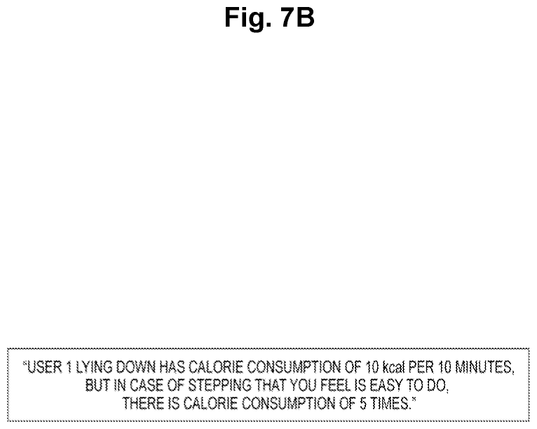

FIG. 7A is a diagram illustrating an example of the message syntax stored in the message syntax database 208. FIG. 7B is a diagram illustrating an example of the message generated by the message generation unit 209. FIG. 7B illustrates an example of a case where the non-recommended action A is “the user 1 is lying down”, the presentation evaluation unit is “10 minutes”, the objective value v_{A} per evaluation unit of the non-recommended action A is “10 kcal”, the evaluation axis is “calorie consumption”, the subjective factor f₀ is “easy to do”, the recommended action B is “stepping”, and the utility is “5 times”. The message generation unit 209 acquires the message syntax illustrated in FIG. 7A stored in the message syntax database 208, and creates a message by inserting the non-recommended action A, the presentation evaluation unit, the objective value v_{A}, the evaluation axis, the subjective factor f₀, the recommended action B, and the utility included in the message creation information into respective portions indicated by [ ] of the message syntax illustrated in FIG. 7A. This message causes the user 1 to grasp the recommended action by the subjective factor f₀ different from the execution positive factor f_{A} that is a factor of selecting the current action, and gives the user 1 a trigger to cause the user 1 to turn one's attention to another way of grasping. Such a message is desirably in a format that enables the user 1 to recognize that the value of the recommended action is greater by making the current action and the recommended action in a comparison format.

The message presentation unit 210 presents the message generated by the message generation unit 209 to the user 1 via the output device 272 of the user interface device 27, and prompts the user 1 to take the recommended action B described in the message (step S106). Note that some sort of emphasized display may be performed, such as setting the font of a portion to be emphasized, such as the utility portion in the message, to be large or changing the color.

[Function and Effect]

It is possible to select a recommended action that is likely to be felt as valuable to the user 1. Then, by presenting the user 1 with a message in which the value of the selected recommended action is replaced with a subjective factor that the user 1 feels has merit, the user 1 can easily practice the recommended action.

### Other Embodiments

Note that the present invention is not limited to the above-described embodiments. For example, in the above embodiments, the example has been described in which increasing the calorie consumption is targeted, but the present invention is also applicable to suppression of calorie intake, suppression of article purchase, and the like. For example, in a case where suppression of expenses due to article purchase or the like is targeted, the objective value v_{B} in step S207 has a value expected to be smaller than the objective value v_{A}.

In addition, the methods described in the above-described embodiments can be stored in a storage medium such as a magnetic disk (floppy (registered trademark) disk, hard disk, or the like), an optical disk (CD-ROM, DVD, MO, or the like), or a semiconductor memory (ROM, RAM, flash memory, or the like) as programs (software means) that can be implemented by a computing machine (computer), or can also be distributed by being transmitted through a communication medium. Note that the programs stored on the medium side also include a setting program for configuring, in the computing machine, a software means (not only an execution program but also tables and data structures are included) to be executed by the computing machine. The computing machine that implements the present device executes the above-described processing by reading the programs stored in the storage medium, constructing the software means by the setting program as needed, and controlling the operation by the software means. Note that the storage medium described in the present specification is not limited to a storage medium for distribution, but includes a storage medium such as a magnetic disk or a semiconductor memory provided in the computing machine or in a device connected via a network.

In short, the present invention is not limited to the above-described embodiments, and various modifications can be made in the implementation stage without departing from the gist thereof. In addition, the embodiments may be implemented in appropriate combination if possible, and in this case, combined effects can be obtained. Furthermore, the above-described embodiments include inventions at various stages, and various inventions can be extracted by appropriate combinations of a plurality of disclosed components.

### REFERENCE SIGNS LIST

1 User
2 User terminal
21 Processor
22 Program memory
23 Data memory
24 Communication interface
25 Input/output interface
26 Bus
27 User interface device
28 Sensor
201 User non-recommended action detection unit
202 Execution positive factor collection unit
203 Recommended action list database
204 Recommended action subjective/objective database
205 Recommended action selection unit
206 Evaluation unit database
207 Recommended action reframing unit
208 Message syntax database
209 Message generation unit
210 Message presentation unit
271 Input device
272 Output device

1. A recommended action selection device comprising:
a processor; and
a storage medium having computer program instructions stored thereon, when executed by the processor, perform to:
detects that a user is taking a non-recommended action;
collects an execution positive factor that is a subjective factor for which the user is taking the non-recommended action; and
selects a subjective factor other than the execution positive factor, acquires a plurality of recommended action possibilities, and selects a recommended action to be recommended to the user on a basis of a first score indicating how easily each of the plurality of recommended action possibilities is implemented by the user for the subjective factor selected, a second score indicating how familiar the user is with each of the plurality of recommended action possibilities, and a first objective value for an evaluation axis for evaluating the plurality of recommended action possibilities.

2. The recommended action selection device according to claim 1, wherein the recommended action is a recommended action possibility that maximizes a sum of the first score weighted, the second score weighted, and the first objective value weighted.

3. The recommended action selection device according to claim 1, wherein the computer program instructions further perform to converts a second objective value indicating the non-recommended action on the evaluation axis and a third objective value indicating the recommended action selected, on the evaluation axis, into an objective value of a presentation evaluation unit, and calculates utility of the recommended action selected, on a basis of the second objective value converted and the third objective value converted.

4. The recommended action selection device according to claim 3, wherein the third objective value has a value expected as compared with the second objective value.

5. The recommended action selection device according to claim 3, wherein the computer program instructions further perform to generates a message to be presented to the user by inserting each of the non-recommended action, the presentation evaluation unit, the third objective value converted, the evaluation axis, the subjective factor selected, the recommended action, and the utility calculated, into message syntax.

6. A recommended action selection method comprising:
detecting that a user is taking a non-recommended action;
collecting an execution positive factor that is a subjective factor for which the user is taking the non-recommended action;
selecting a subjective factor other than the execution positive factor;
acquiring a plurality of recommended action possibilities; and
selecting a recommended action on a basis of a first score indicating how easily each of the plurality of recommended action possibilities is implemented by the user for the subjective factor selected, a second score indicating how familiar the user is with each of the plurality of recommended action possibilities, and a first objective value for an evaluation axis for evaluating the plurality of recommended action possibilities.

7. A non-transitory computer-readable medium having computer-executable instructions that, upon execution of the instructions by a processor of a computer, cause the computer to function as the recommended action selection device according to claim 1.
